(19) United States
(12) Patent Application Publication
(10) Pub. No.: US 2024/0325679 A1
(43) Pub. Date: Oct. 3, 2024

(54) LOW-BLUE LIGHT SOURCE

(71) Applicant: KORRUS, INC., LOS ANGELES, CA (US)

(72) Inventors: Paul Kenneth PICKARD, LOS ANGELES, CA (US); Robert Harris, LOS ANGELES, CA (US); Qinghua Zeng, LOS ANGELES, CA (US)

(21) Appl. No.: 18/739,080

(22) Filed: Jun. 10, 2024

Related U.S. Application Data
(63) Continuation of application No. PCT/US22/52542, filed on Dec. 12, 2022.
(60) Provisional application No. 63/288,419, filed on Dec. 10, 2021.
(60) Provisional application No. 63/291,000, filed on Dec. 17, 2021.

(51) Int. Cl. A61M 21/02; A61M 21/00; H01L 33/50

(52) U.S. Cl. CPC A61M 21/02; H01L 33/504; A61M 2021/0044; A61M 2205/587

(57) ABSTRACT

A light source comprising a pump LED for emitting a pump light having a peak wavelength of 420 to 430 nm, one or more wavelength converting materials configured for absorbing a portion of said pump light and converting said portion to converted light, and wherein emitted light is a combination of said converted light and said pump light, said emitted light having a first SPD between 380 and 780 nm having a first power, a second SPD between 440 and 490 nm having a second power, wherein said second power is no greater than 2% of said first power, and wherein said emitted light has a CRI of at least 85, an Rf of at least 60 a CIE whiteness of at least 70.

### CROSS REFERENCE TO RELATED APPLICATIONS

The present patent application is a continuation of International Application No. PCT/US22/52542, filed Dec. 12, 2022, which claims the benefit of U.S. Provisional Patent Application 63/288,419, filed Dec. 10, 2021, and U.S. Provisional Patent Application 63/291,000, filed Dec. 17, 2021, the entire disclosures of each are hereby incorporated herein by reference.

### FIELD OF INVENTION

This disclosure relates to the field of illumination products, and, more particularly, to apparatus and methods for providing circadian-friendly LED light sources.

### BACKGROUND

Identification of non-visual photoreceptors in the human eye (so-called intrinsically photosensitive retinal ganglion cells, or “ipRGCs”) linked to the circadian system has sparked considerable interest in the effects of various light spectra on health and amenity for human beings. High circadian stimulation may lead to positive effects such as resetting sleep patterns, boosting mood, increasing alertness and cognitive performance, and alleviating seasonal affective depression. However, mis-timed circadian stimulation can also be associated with disruption of the internal biological clock and melatonin suppression, and may be linked to illnesses such as cancer, heart disease, obesity, and diabetes.

Circadian stimulation is associated with glucocorticoid elevation and melatonin suppression and is most sensitive to light in the blue wavelength regime. With the preponderance of light-emitting diode (LED) illumination products being based on blue-primary phosphor-converted white-emitting LEDs, the situation has developed that most LED-based illumination sources have higher levels of circadian stimulation than the traditional sources they are intended to replace.

Of particular interest herein is the emission of the blue primary color, which has a peak emission around 450-480 nm.

As described in U.S. Pat. No. 9,915,775, Applicants have discovered that a spectrum can be configured to appear substantially white, despite a substantial absence of blue radiation, For simplicity, such spectra is referred to herein as “blue-free.” Blue-free emitters are desirable due to their reduced impact on the human circadian cycle, which is important for instance, in the evening before going to sleep.

The conventional low-blue lamp was designed around a GaN on GaN violet chip so there was initially limited flexibility in selecting the pump wavelength. This conventional violet pump wavelength is about 412 nm cold/416 nm hot. That chip was paired with a commercially-available beta-SiON phosphors to minimize the blue/cyan emission. The result was an LED with minuscule blue content, but relatively poor color rendering index (CRI) and Rf values. CRI is a measurement of how natural colors render under an artificial white light source when compared with sunlight, and Rf is an index that measures the fidelity of a light source to its reference source. Such blue free light makes rendering many colors and nominally white materials very difficult if not impossible.

Therefore, there is a need to significantly improve the color rendering of blue free lights while maintaining a meaningful degree of circadian friendliness. The present invention fulfills this need among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

An important aspect of the present invention is using a pump LED having a slightly longer wavelength than conventional violet pumps used in circadian friendly lighting. By using a slightly longer wavelength certain unexpected benefits were realized.

First, Applicant recognized that using a longer wavelength violet pump allowed the violet peak power to be reduced. Specifically, in the region of interest (400-440 nm), the human eye is more sensitive to longer wavelengths. (This can also be understood by studying the blue curve (z) in the CIE 1931 CMFs. Peak sensitivity is around 440 nm and falls off in either direction.) Thus, less violet content is needed if the wavelength is longer.

Second, the longer wavelength of the violet pump also significantly improves the color rendering of the light because many colors have strong reflectivity in the blue region and poor reflectivity in the violet region. It is impossible to accurately render these colors if the light source does not contain any blue photons. So moving the violet peak closer to blue helps in this respect.

Third, Applicant found that the emission power of the longer wavelength violet pump drops off sharply after the peak thus minimizing bleed into the short blue wavelengths.

Fourth, Applicant found that the longer wavelength of the violet pump was long enough to allow conventional, blue-pumped phosphors to be used, such as GAL. By using a conventional green phosphor such as GAL, which has a broad emission spectrum, small amounts of long blue and cyan light are emitted which greatly improves the CRI.

Fifth, Applicant found that the slightly longer wavelength violet pump preferentially pumps phosphors such that the phosphors can be selected to enhance the emission spectrum of the light. For example, in one embodiment, GAL phosphor is used, which preferentially absorbs longer wavelength violet. Alternatively, in one embodiment, Beta-SiON is used, which preferentially absorbs shorter wavelength violet. In other words, GAL has higher excitation and absorption efficiency in longer violet wavelengths, and beta-SiON has higher excitation and absorption efficiency in shorter violet wavelengths

Sixth, Applicant found that even though the violet pump had a slightly longer wavelength, the incremental lengthening had little impact on the circadian friendliness of the light.

Accordingly, in one embodiment, the present invention relates to a light source for emitting low blue light. In one embodiment, the present invention relates to a circadian-friendly light source for emitting emitted light, the light source comprising: (a) pump LED for emitting a pump light having a peak wavelength of 420 to 430 nm; (b) one or more wavelength converting materials configured for absorbing a portion of the pump light and converting the portion to converted light; (c) wherein the emitted light is a combination of the converted light and a second portion of the pump light which is not absorbed by the one or more wavelength converting materials, the emitted light having a first spectral power distribution (SPD) between 380 and 780 nm having a first power, a second SPD between 440 and 490 nm having a second power, and a third SPD between 380 and 440 nm having a third power, wherein the second power is no greater than 2% of the first power, and wherein the emitted light has a CRI of at least 85, and an Rf of at least 60.

In one particular embodiment, the light source of the present invention significantly improves the color rendering over conventional low-blue displays while maintaining a meaningful degree of circadian-friendliness. To do this, Applicant further increased the pump wavelength to 424 nm cold/428 nm hot along with a broader green phosphor. This type of green phosphor is traditionally used with a 450 nm die in high CRI white and warm-white applications, although Applicant found it performed well with a slight longer violet.

In an alternative embodiment, the light source of the present invention improves the circadian-friendliness compared to other embodiments. To this end, Applicant combines the green phosphor of the other embodiment with a narrower green phosphor that is very similar to the one used in conventional low blue light. In yet another embodiment, rather than a mixture of phosphors, a single green phosphor (possibly a longer GAL or a Green YAG) may be used.

### BRIEF DESCRIPTION OF FIGURES

FIG. 1 shows a comparison of the spectrums of a conventional low blue light, a modified conventional low blue light, one embodiment of the low blue light of the present invention, and an alternative embodiment of the low blue light of the present invention.

FIG. 2(a)-(c) are tables showing configuration, performance, color rendering, whiteness rendering, and circadian friendliness for different embodiments of the light source of the present invention.

### DETAILED DESCRIPTION

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

In one embodiment, invention relates to a circadian-friendly light source for emitting emitted light, the light source comprising: (a) pump LED for emitting a pump light k, having a peak wavelength of 420 to 430 nm; (b) one or more wavelength converting materials configured for absorbing a portion of the pump light and converting the portion to converted light; (c) wherein the emitted light is a combination of the converted light and a second portion of the pump light which is not absorbed by the one or more wavelength converting materials, the emitted light having a first SPD between 380 and 780 nm having a first power, a second SPD between 440 and 490 nm having a second power, and a third SPD between 380 and 440 nm having a third power, wherein the second power is no greater than 2% of the first power, and wherein the emitted light has a CRI of at least 85, and an Rf of at least 60.

### Pump

In one embodiment, the LED pump has a peak (measured at operating temperature) between 420 nm and 430 nm, or between 425 nm and 430 nm, or between 427 nm and 429 nm. In one particular embodiment, the pump has a peak at 428 nm. As mentioned above, an unexpected benefit of using a violet pump having a peak wavelength in this range is the relatively sharp fall from the peak on the right side (i.e. longer wavelengths). That is, sharp fall means that the emitted from the pump LED does not bleed significantly into the short blue range. Additionally, by using a violet pump having a peak wavelength that is longer than a conventional violet pumps used in low blue applications, conventional phosphors can be used.

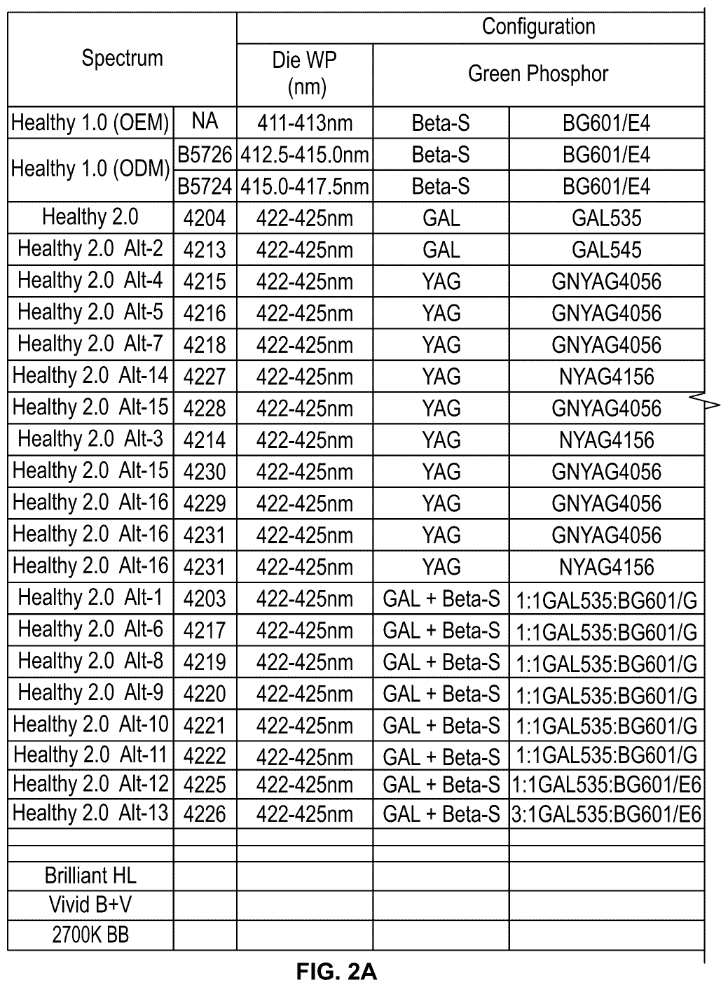

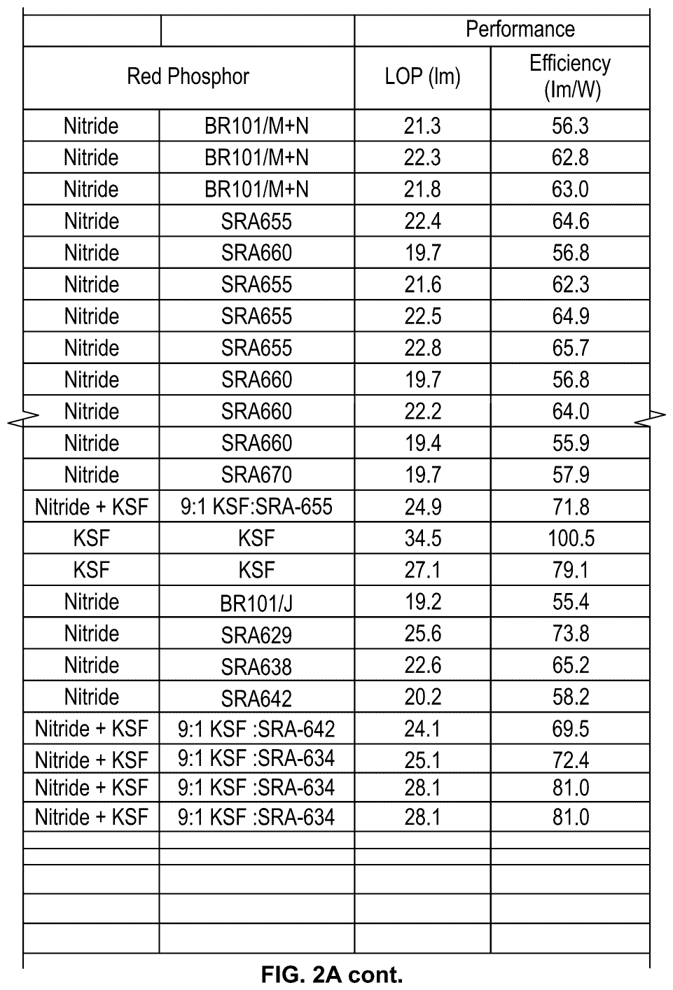

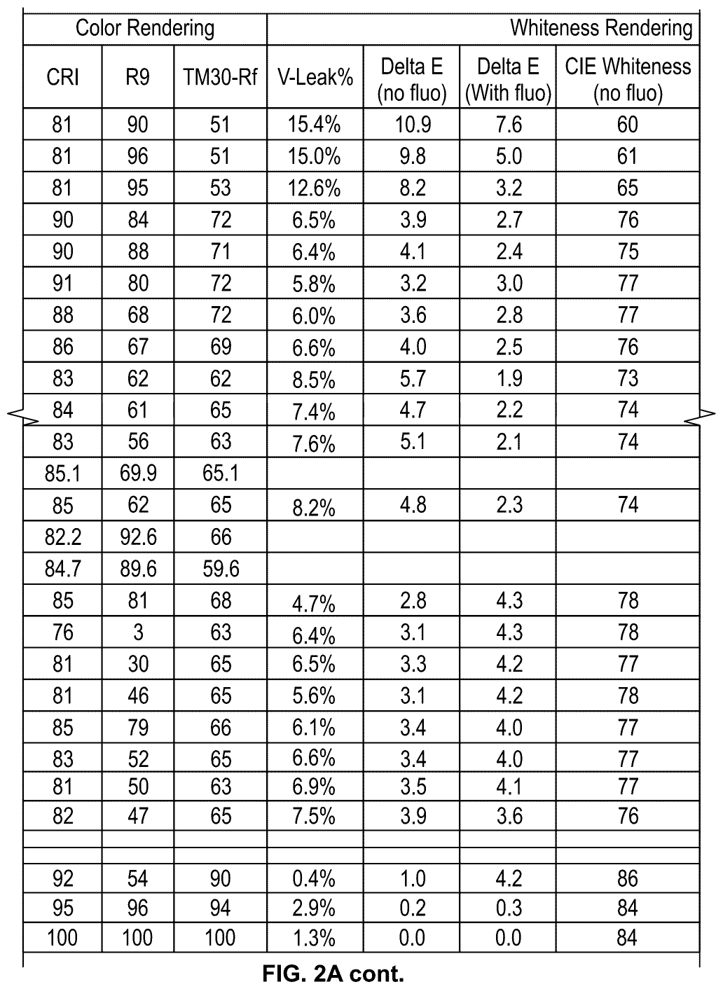

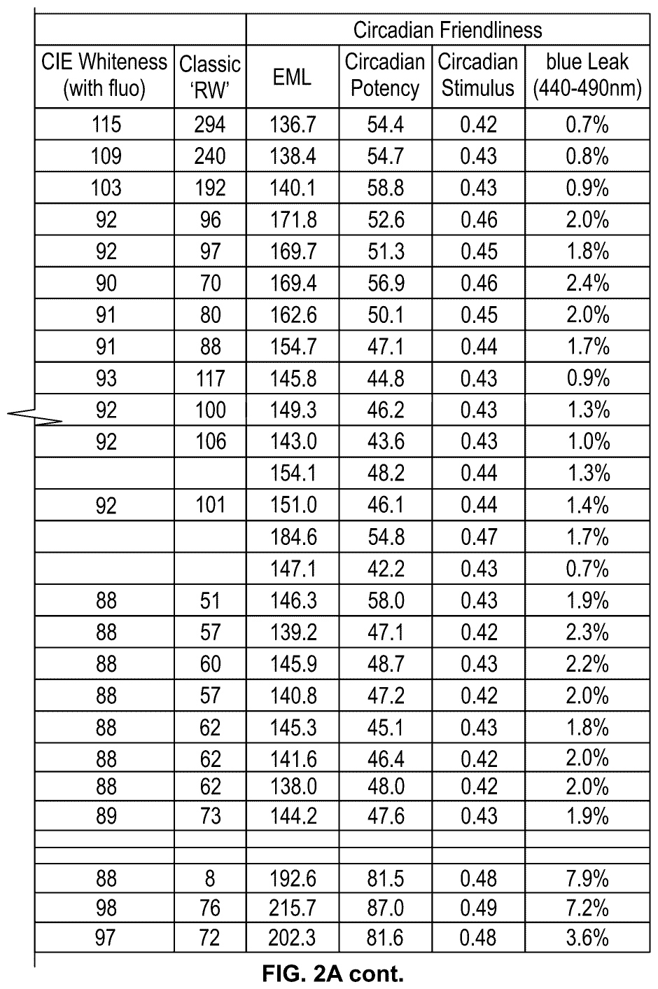

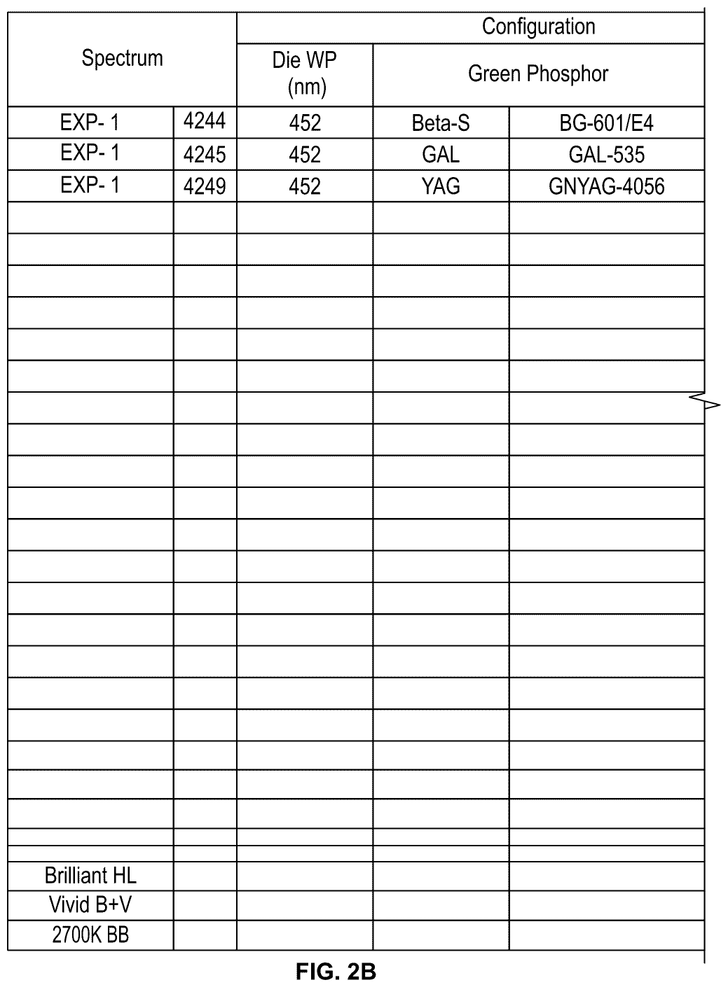

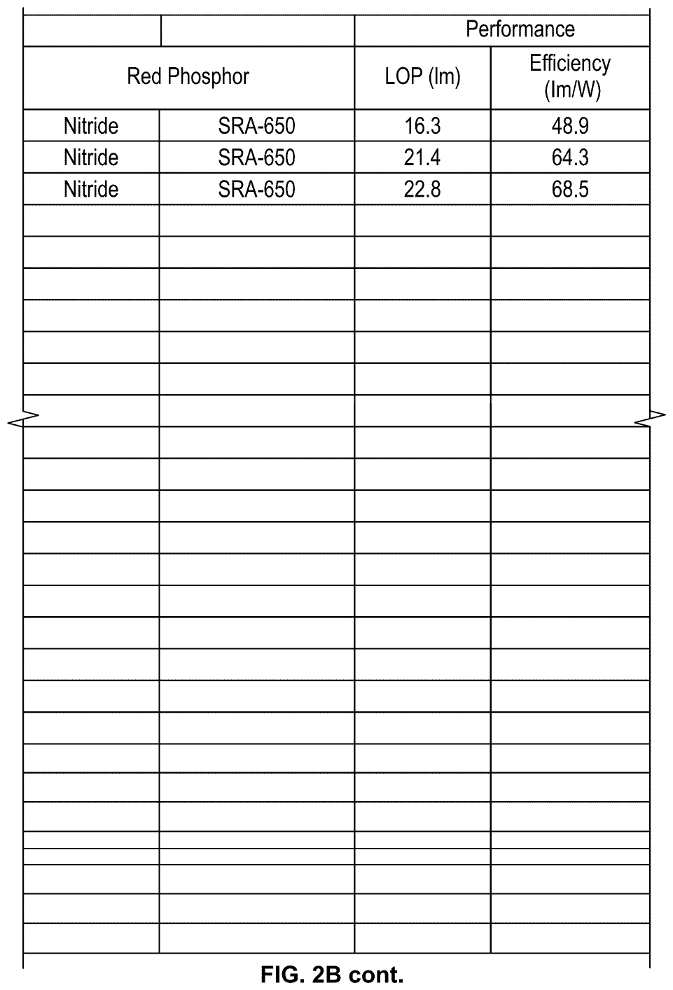

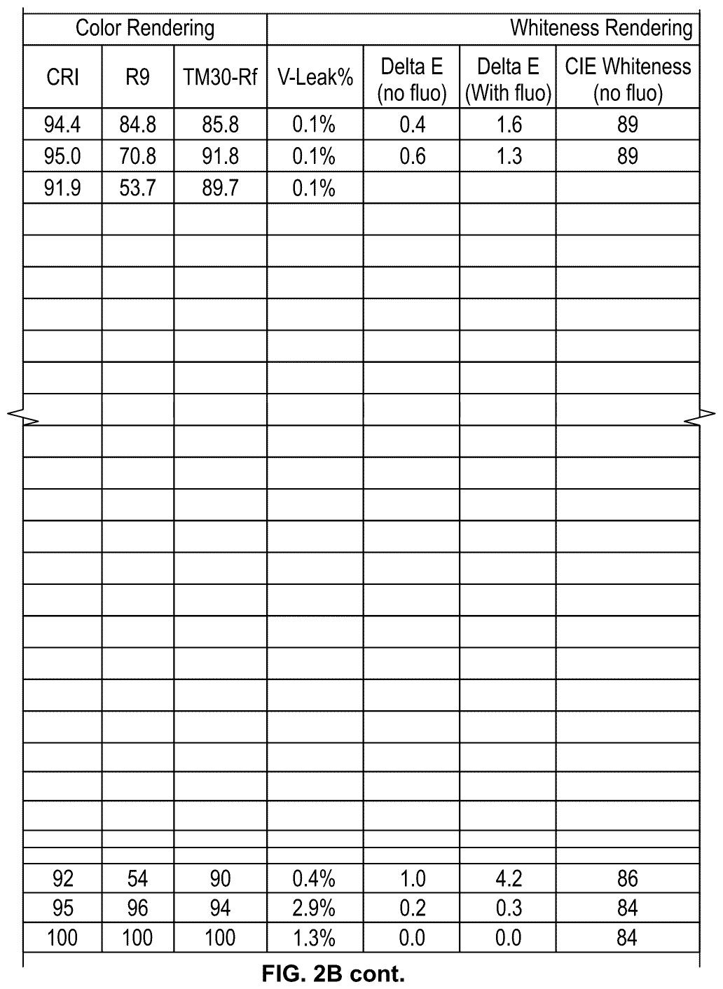

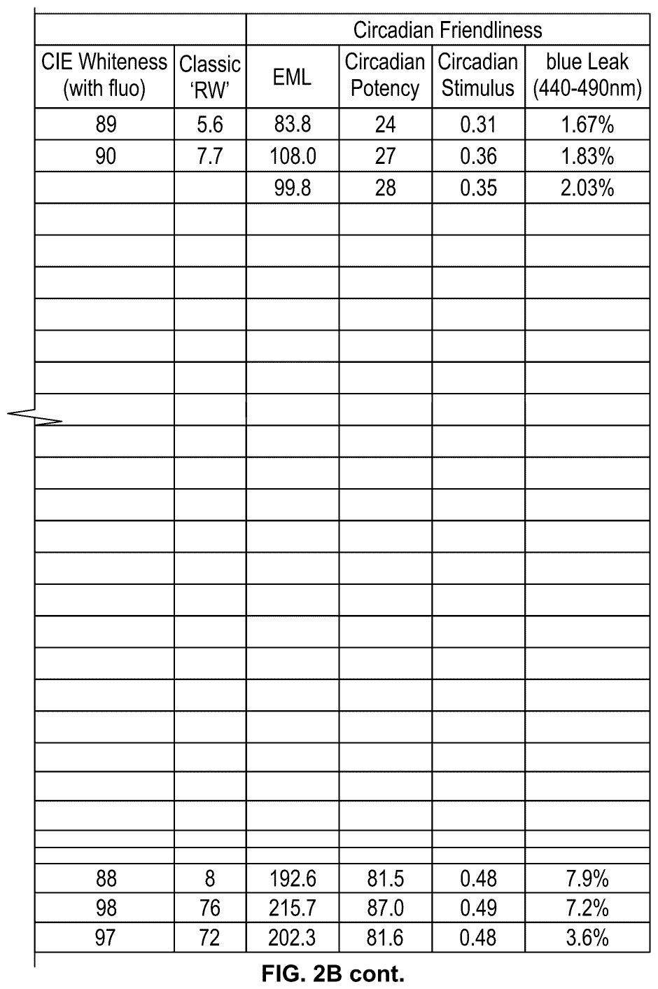

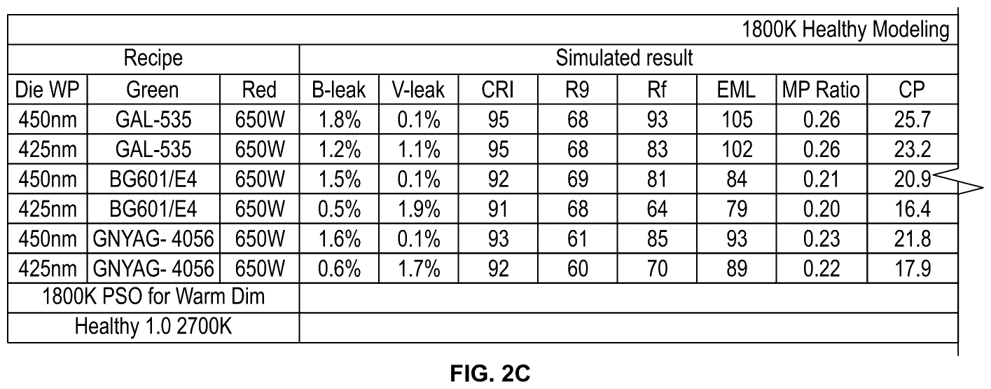

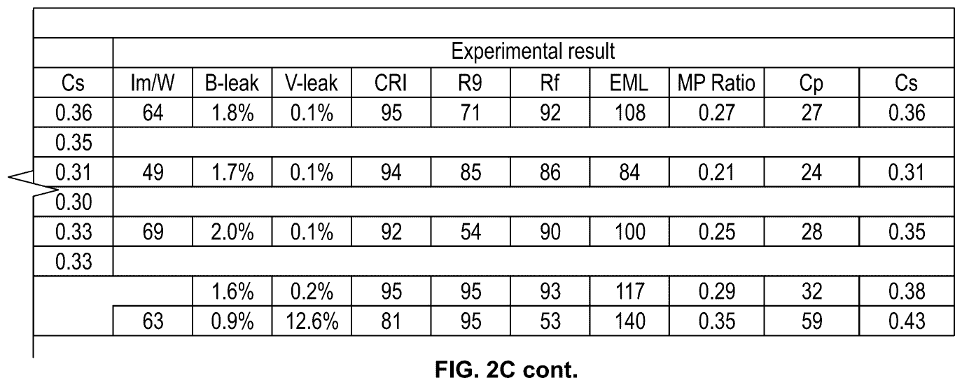

In one embodiment, a second pump having a longer wavelength is added to the light source to reduce the amount of violet leak. For example, in one embodiment, a 450 nm pump is added to reduce the reliance on the violet pump. FIG. 2c shows results of various embodiments having both a 425 nm and a 450 nm pump.

### Phosphors

In one embodiment, at least one of the phosphors is a green phosphor. In one embodiment, the green phosphor is chosen to increase light quality. To that end, generally a green phosphor having a wider emission spectrum is preferred. For example, in one embodiment, the green phosphor comprises GAL Green or yellow aluminate. This phosphor has a relatively broad emission spectrum such that it emits a small amount of long blue and cyan light to improve the quality of light. In a particular embodiment, the GAL Green or yellow aluminate phosphor is INTX GAL-535. It has been found that the GAL phosphor preferentially absorbs longer wavelength violet. In other words, it pulls from the right side of the violet peak. This type of green phosphor is commonly used with a 450 nm die in high CRI white and warm-white applications.

In embodiments in which lower circadian stimulation is desired over light quality, it may be preferable to modify the green phosphor to have a narrower emission spectrum. For example, in one embodiment, at least one of the phosphors is GAL plus Beta-SiON. In a particular embodiment, the green phosphor comprises a mix of INTX GAL-535 and MCC BG-601/G. It has been found that the Beta-SiON tends to dominate in this phosphor mix, and preferentially absorbs shorter wavelength violet. In other words it pulls from the left side of the violet peak. Still other free phosphors will be obvious to those of skill in the art in light of this disclosure. For example, in one embodiment, a longer GAL or a green YAG may be used, or YAG plus Beta-SiON Blend. The use of YAG and be beneficial because absorbs violet and blue in more even proportions compared to GAL, which absorbs primarily blue but very little violet.

In one embodiment, the light source also comprises a red phosphor. Again, those of skill he art in light of this disclosure can determine, without undue extermination, optimum red phosphors. In one embodiment, red phosphors a nitride, for example, MCC BR-101-SR11OR, INTX SRA-655, or MCC BR-101/J. Alternatively, the red phosphor may comprise, for example, KSF.

### Spectrum

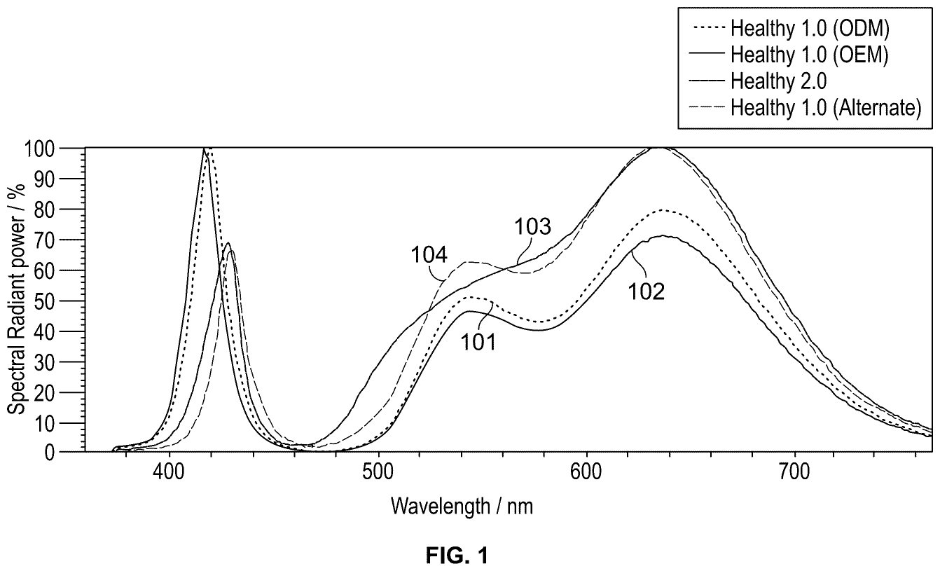

Referring to FIG. 1, a comparison is shown of the spectrums of a conventional low blue light 101, a modified conventional low blue light 102, one embodiment of the low blue light of the present invention 103, and an alternative embodiment of the low blue light of the present invention 104.

The Correlated Color Temperature (CCT) of the light can vary with the application. Generally a CCT of between 1500 and 6500k is preferred. In one embodiment, the CCT is less than 5000K, or less than 4000K, or less than 3000k, or is about 2700K or about 1800K.

### Spectral Power Distribution (SPD)

In one embodiment, the third power is no greater than 10%, or no greater than 8%, no greater than 6%, no greater than 5% of the first power. In one embodiment, the second power is no greater than 2%, or no greater than 1.5%, or no greater than 1% of the first power.

### Circadian Friendliness

Circadian effect may be measured in different ways, including, for example, circadian potency (CP), circadian stimulus (CS), and Equivalent Melanopic Lux (EML). CP is calculated by linear projection of the spectrum on the CP efficiency curve as below.

$$C P = 832.41 \times {\int }_{{λ}_{min}}^{{λ}_{max}} S P D ( λ ) \cdot {V}_{C P} ( λ ) \cdot d λ$$

Here, SPD is the source spectrum and Vcp is the efficiency curve described above. Circadian Stimulus (CS). In one embodiment, CP is no greater than 54, or is no greater than 53, or is no greater than 53, or is no greater than 52, or is no greater than 51, or is no greater than 50. CS is a transformation of circadian light into relative units, from zero (the threshold for circadian system activation) to 0.7 (response saturation), and is directly proportional to nocturnal melatonin suppression after one hour of light exposure (zero to 70 percent). In one embodiment, CS is no greater than 0.50, or is no greater than 0.47, or is no greater than 0.46, or is no greater than 0.45, or is no greater than 0.43. In one embodiment, the Equivalent Melanopic Lux (EML) is no greater than 180, or is no greater than 170, or is no greater than 160, or is no greater than 150.

### Quality

In one embodiment, CRI is at least 82, or is at least 85, or is at least 87, or is at least 88, or is at least 89, or is at least 90. In one embodiment, R9 is at least 60, or is at least 65, or is at least 70, or is at least 75, or is at least 80, or is at least 85 In one embodiment, TM30-Rf is at least 55, or is at least 60, or is at least 65, or is at least 70. In one embodiment, Classix Rw is no greater than 150, or is no greater than 125, or is no greater than 115, or is no greater than 100.

The CIE measure of whiteness is a measurement of the light reflected by the paper across the visible (daylight) spectrum. The CIE have set a standard of D65 illumination which is a standard representation of outdoor daylight under which the amount of light reflected is measured. Here, Applicant measured the perceived adapted whiteness of 8 materials under the test illuminant. CIE whiteness without fluorescence of at least 70, or at least 72, or at least 73, or at least 74, or at least 75, or at least 76, or at least 77, or at least 78.

### Efficiency

In one embodiment, the light source has an efficiency (Lm/W) of at least 55, or at least 60, or at least 65, or at least 70.

When optimizing the various variables of the light source of the present invention, such as, pump peak wavelength, phosphors, CP, EML, and light quality, Applicant, in one embodiment, attempts to balance the following performance goals:
improve TM-30 Rf from its current low 50s to at least mid 60s, with 70+ desired
minimize the Violet Leak and the fluorescent lighting effect
   Conventional low-blue lamp has an Rw between 192-240 vs. embodiments of the light of the present invention that range from 57-117
Improve efficacy
Try to maintain R9 as close to 90 as possible, but not to the detriment of TM-30 Rf

### Examples

As mentioned above, an important aspect of the present invention is using a pump LED having a slightly longer wavelengths than traditional violet pumps used in circadian friendly lighting. Moreover, as mentioned above, by using a slightly longer wavelengths violet pump certain unexpected benefits were realized. For example, Applicant discovered that a slight increase in wavelength has a beneficial impact on the response (i.e., absorption/emission) of various phosphors. Referring to FIGS. 2A-C, a matric of different phosphor formulations are presented with the longer wavelengths violet LED pump. FIG. 2a is a comparison of convention low blue light (rows 3-5) to various embodiments of the low blue light of the present invention (rows 6-25) at a color temperature of 2700K. Specifically, GAL Green+Nitride Red provides good color rendering (90+ CRI) and efficiency but relatively high EML. Modulating the wavelength of the GAL has only a very weak effect on circadian metrics. YAG (Green)+Nitride Red is versatile as the YAG wavelength can be tuned to deliver good color rendering (90+ CRI) or good circadian metrics (EM<150), although not necessarily at the same time. Increasing the wavelength of the YAG improves the circadian metrics but reduces the color rendering and efficiency metrics. (GAL+Beta Sialon) Green+Nitride Red is also quite versatile. The GAL:Beta Sialon ratio can be adjusted—GAL-rich has better color rendering and Beta Sialon-rich has better circadian metrics. (GAL+Beta Sialon) Green+(Nitride+KSF) Red: using KSF/PSF narrow red phosphor improves the efficiency. The 85 CRI/80 R9/65Rf version is very attractive and likely 20-30% more efficient than a conventional low blue light.

Noteworthy: (1) almost all the configurations have more blue leak than Conventional low-blue lamp (4227 is the only non-KSF recipe to equal that percentage); (2) despite the increased blue leak, the Circadian Potency of 4228 is nearly 20% lower than Healthy 1.0; (3) almost ALL the variants have a CP lower than Healthy 1.0, even with blue leak ranging from 0.9% to 2.3%; (4) the EML for the variants is either equal to or higher than Conventional low-blue lamp (This is reflective of the additional green coverage—one of the ways we're boosting TM-30 Rf)

FIG. 2b shows performance data for alternative embodiments of the present invention at 1800K. FIG. 2c shows the effect of adding a second 450 nm pump to various embodiments having a 425 nm pump to reduce violet leak at a color temperature of 1800K.

These and other advantages may be realized in accordance with the specific embodiments described as well as other variations. It is to be understood that the above description is intended to be illustrative, and not restrictive. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled.

What is claimed is:

1. A circadian-friendly light source for emitting emitted light, said light source comprising:
a pump LED for emitting a pump light having a peak wavelength of 420 to 430 nm;
one or more wavelength converting materials configured for absorbing a portion of said pump light and converting said portion to converted light; and
wherein said emitted light is a combination of said converted light and a second portion of said pump light which is not absorbed by said one or more wavelength converting materials, said emitted light having a first SPD between 380 and 780 nm having a first power, a second SPD between 440 and 490 nm having a second power, and a third SPD between 380 and 440 nm having a third power, wherein said second power is no greater than 2% of said first power, and wherein said emitted light has a CRI of at least 85, an Rf of at least 60 a CIE whiteness of at least 70.

2. The light source of claim 1, wherein said one or more converting materials also comprises at least one red phosphor.

3. The light source of claim 1, wherein said one or more converting materials also comprises at least KSF.

4. The light source of claim 1, wherein said third power is no greater than 10% (or 8%, 6%, 5%) of said first power.

5. The light source of claim 1, wherein said CRI is at least 87, or at least 90.

6. The light source of claim 1, wherein said Rf is at least 65, or at least 70.

7. The light source of claim 1, wherein said CIE whiteness of at least 73, or at least 75.

8. The light source of claim 1, wherein said absorption peak for said at least one green phosphor is greater than 430 nm.

9. The light source of claim 8, wherein said at least one green phosphor comprises a GAL

10. The light source of claim 1, wherein said at least one green phosphor comprises a first phosphor and a second phosphor.

11. The light source of claim 10, wherein said second green phosphor absorb pump light at a shorter wavelength than said first green phosphor.

12. The light source of claim 11, wherein said first green phosphor is GAL and said second green phosphor is Beta Sialon.

13. The light source of claim 8, wherein said at least one green phosphor comprises a YAG.

14. The light source of claim 13, wherein said at least one green phosphor comprises a first phosphor and a second phosphor.

15. The light source of claim 14, wherein said first green phosphor is YAG and said second green phosphor is Beta Sialon.

16. The light source of claim 1, wherein said at least one red phosphor comprises nitride.

17. The light source of claim 1, further comprising a second pump LED having a peak wavelength greater than 430 nm.

18. The light source of claim 17, wherein said second pump LED has a peak wavelength of 440-460 nm, or 445-455 nm, or 450 nm.

19. The light source of claim 1, wherein said pump LED has a peak wavelength of 425-430 nm, or 427-429 nm, or 428 nm.
